(19) United States
(12) Patent Application Publication
(10) Pub. No.: US 2023/0310399 A1
(43) Pub. Date: Oct. 5, 2023

(54) COMBINATORIAL THERAPEUTIC SYSTEMS AND METHODS THAT INCLUDE SYSTEMIC, CENTRALLY AND PERIPHERALLY ACTING ANALGESICS

(71) Applicant: COGNIFISENSE, INC., Sunnyvale, CA (US)

(72) Inventors: Tassilo BAEUERLE, Sunnyvale, CA (US); Harald STOCK, (US)

(21) Appl. No.: 18/012,058

(22) PCT Filed: Jul. 22, 2021
(86) PCT No.: PCT/US2021/042848

§ 371 Date: Dec. 21, 2022

Related U.S. Application Data
(60) Provisional application No. 63/056,339, filed on Jul. 24, 2020.

(51) Int. Cl. A61K 31/4436; A61K 31/445; A61K 31/5415; A61P 25/04; G16H 20/70

(52) U.S. Cl. CPC A61K 31/4436; A61K 31/445; A61K 31/5415; A61P 25/04; G16H 20/70

(57) ABSTRACT

Combinatorial therapeutic systems can include a systemic, centrally and peripherally acting analgesics and an artificial sensory experience. Methods for treating and/or mitigating pain can include receiving a systemic, centrally and peripherally acting analgesic and an artificial sensory experience. The analgesic can include oliceridine or bupivacaine-meloxicam. The artificial sensory experience can include one or more of 2D/3D/4D artificial sensory experiences, holography, augmented reality, virtual reality, and/or mixed reality. In some instances, the virtual reality is virtual reality neuropsychological therapy or virtual reality distraction therapy.

### BACKGROUND

Pain affects virtually all humans at some point in their lives. In particular, chronic pain is a major contributor to disability in the industrialized world and is the cause of an unquantifiable amount of suffering. However, chronic pain is more than a series of nociceptive or acute pain events. It is a complex condition consisting of physiological and psychological components. In fact, neuroimaging research has shown that chronic pain is associated with parts of the brain different from those associated with acute pain. Thus, to provide lasting relief from chronic pain therapies should address both the physiological and the psychological components of chronic pain.

Commonly prescribed analgesics for pain include opioid analgesics, which carry significant risk of side effects and/or addiction and have responder rates as low as 30%. Some opiates show only marginal incremental pain reduction versus placebo. This means these pharmacological treatments offer limited effectiveness for a large proportion of chronic pain sufferers and carry significant risks. Moreover, these opioids, and most other analgesics, do not address the underlying; e.g., psychological components of chronic pain; they simply mask the pain for a time.

The need for an effective (chronic) pain therapy to address both the physical and the psychological components of (chronic) pain speaks to a long-felt (but unmet) need for additional therapies. Needed therapies include those that not only treat and/or mitigate (chronic) pain, but also those that may reduce risk of drug tolerance buildup, side effects, and addiction.

### BRIEF SUMMARY

Embodiments described herein are directed to combinatorial therapeutic systems and methods for treating or mitigating pain. An exemplary combinatorial therapeutic system includes (i) a centrally and peripherally acting analgesic having systemic exposure and (ii) an artificial sensory experience. The analgesic can be, preferably, oliceridine and/or bupivacaine-meloxicam. Other embodiments can additionally or alternatively include other analgesic drugs that have a similar mechanism of action to oliceridine and/or bupivacaine-meloxicam. Oliceridine is an opioid receptor agonist selective for the μ-opioid receptor. It is also a G protein pathway selective modulator. Bupivacaine-meloxicam is a combination drug that functions as a prostaglandin-mediated non-steroidal anti-inflammatory drug (NSAID). Any of the foregoing analgesic selections can be combined with an artificial sensory experience selected from 2D artificial sensory experience, 3D artificial sensory experience, 4D artificial sensory experience, holography, augmented reality, virtual reality, or mixed reality. In some embodiments, the virtual reality is virtual reality neuropsychological therapy (VRNT). In some embodiments, the virtual reality is virtual reality distraction therapy. In some embodiments, multiple types of artificial sensory experiences (e.g., distraction therapy and VRNT) can be utilized together.

Methods for treating and/or mitigating pain include receiving an analgesic selected from oliceridine or bupivacaine-meloxicam. Any of the disclosed method acts of receiving an analgesic can be combined in a combinatorial therapy with receiving an artificial sensory experience. Receiving the artificial sensory experience can include receiving one or more of 2D artificial sensory experience, 3D artificial sensory experience, 4D artificial sensory experience, holography, augmented reality, virtual reality, or mixed reality. In some variations the sensory experience can be configured to visualize and/or virtually ameliorate the patient's pain; in some variations, this can be done according to the patient's own experience of that pain. In some embodiments, receiving the artificial sensory experience includes receiving psychological or physical training. In some variations, the artificial sensory experience is virtual reality, preferably VRNT, virtual reality distraction therapy, or both.

Methods for treating and/or mitigating pain, particularly for treating and/or mitigating post-operative pain, include receiving oliceridine and/or bupivacaine-meloxicam. These methods can additionally include the method act of receiving an artificial sensory experience. The artificial sensory experience can be any of 2D artificial sensory experience, 3D artificial sensory experience, 4D artificial sensory experience, holography, augmented reality, virtual reality, or mixed reality. In some variations, the artificial sensory experience is virtual reality, preferably VRNT, virtual reality distraction therapy, or both.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be apparent to one of ordinary skill in the art from the description or may be learned by the practice of the teachings herein. Features and advantages of embodiments described herein may be realized and obtained by means of the instruments and combinations particularly pointed out in the appended claims. Features of the embodiments described herein will become more fully apparent from the following description and appended claims.

### BRIEF DESCRIPTION OF THE DRAWINGS

To further clarify the above and other features of the embodiments described herein, a more particular description will be rendered by reference to the appended drawings. It is appreciated that these drawings depict only examples of the embodiments described herein and are therefore not to be considered limiting of its scope. The embodiments will be described and explained with additional specificity and detail through the use of the accompanying drawings in which:

FIG. 1 is a schematic of an exemplary combinatorial therapeutic system that combines administration of an analgesic and an artificial sensory experience to a patient;

FIG. 2 illustrates an exemplary graph of a patient's pain over time with respect to the concentration of systemic, centrally and peripherally acting analgesic over time that is administered in a combinatorial therapeutic according to the present disclosure;

FIG. 3 illustrates an exemplary graph of a patient's pain over time with respect to the number of doses per day of a systemic, centrally and peripherally acting analgesic over time that is administered in a combinatorial therapeutic according to the present disclosure;

FIG. 4A illustrates an exemplary graph of a patient's pain over time with respect to the efficacy of a systemic, centrally and peripherally acting analgesic, as included within a combinatorial therapeutic according to the present disclosure;

FIG. 4B illustrates an exemplary graph of a patient's pain over time with respect to the efficacy of an artificial sensory experience over time, as included within a combinatorial therapeutic according to the present disclosure;

FIGS. 5A-5C illustrate exemplary graphs of potential effects of combinatorial therapeutic systems/methods employing oliceridine and VRNT on a patient's amount of pain over time; and

FIGS. 6A and 6B illustrate exemplary graphs of potential effects of combinatorial therapeutic systems/methods employing a systemic, centrally and peripherally acting analgesic and VRNT on a patient's response to VRNT.

### DETAILED DESCRIPTION

### Introduction

Pain, and especially chronic pain, is a leading cause of morbidity, and analgesics such as opioids are prevalent in prescription regimens for managing pain. These opioids have responder rates of as little as 30%, and even novel analgesics like tapentadol have been reported as having limited efficacy beyond placebo. At the same time, many opioid analgesics have serious potential side effects and may be addictive. This can lead to abuse. Moreover, regular administration of an opioid analgesic often leads to increased tolerance, reducing the efficacy of the treatment over time. Finally, these and many other analgesics act mostly to mask or temporarily diminish physiological aspects of the experience of pain but fail to address the important underlying psychological or neuropsychological components of pain. Consequently, there is a need for new systems and methods for treating and/or mitigating pain.

As noted above, the embodiments described herein are generally directed to combinatorial therapeutic systems and methods for treating or mitigating pain. It has been surprisingly found that not all analgesics can be used in combinatorial therapies to the same effect. Indeed, it is now appreciated that systemic, centrally and peripherally acting analgesics such as oliceridine and bupivacaine-meloxicam (and/or other drugs with similar mechanisms of action and/or similar intended use), when combined with an artificial sensory experience, provide one or more benefits over the use of these analgesics or artificial sensory experiences alone. This complements previous findings by the same inventors that pain can be treated, and in some cases better managed, using combinatorial therapies that include an artificial sensory experience combined with a systemic, centrally and peripherally acting analgesic.

Embodiments described herein are designed to overcome at least some of the limitations of prior pain treatment regimens by creating a method and/or system that is capable of treating and/or mitigating pain without sole reliance on analgesic therapies, and does so in a low cost, personalized, and repeatable way.

Embodiments described herein could additionally reduce the amount and/or frequency of systemic, centrally and peripherally acting analgesics administered for pain management and may, in some instances, additionally or alternatively, reduce the severity and/or frequency of severe acute pain, episodic, or chronic pain. Additionally, or alternatively, aspects of the present disclosure may provide a synergistic benefit, increasing the efficacy and/or duration of the salubrious effects of systemic, centrally and peripherally acting analgesics. Additionally, or alternatively, embodiments described herein may increase the efficacy of the artificial sensory experience, accelerate the pain reduction, extend the analgesic effect of the artificial sensory experience, and/or reduce the amount of training or use of the artificial sensory experience administered before analgesic effects are realized.

Further, prophylactic treatment regimens can be initiated to decrease the intensity of the pain and/or the duration of the pain.

In some instances, treatment, prophylactic or otherwise, may eliminate any perception of pain or allow the patient to experience the pain with diminished (or without) negative psychological associations. Such embodiments can beneficially enable patients that, for example, experience chronic or persistent episodic pain to curb the perceived intensity and/or the duration of pain with less medication (i.e., in frequency and/or amount), or improve comorbidities associated with chronic pain, such as abnormal sensations in the affected area (e.g., numbness, tingling, prickling, “non-normalness”), disability/reduced function, depression, mood, fatigue/sleep deprivation, fear, and anxiety. Such implementations can measurably increase the patient's quality of life and reduce addiction and reliance on high levels of medications, among other benefits. An improvement in these comorbidities would be of great benefit to many patients, even if there is not reduction in pain intensity. Thus, the present invention would be valuable/efficacious in the instances it treats comorbidities associated with chronic pain, even if there is no reduction in pain intensity.

### Exemplary Combinatorial Treatment Systems

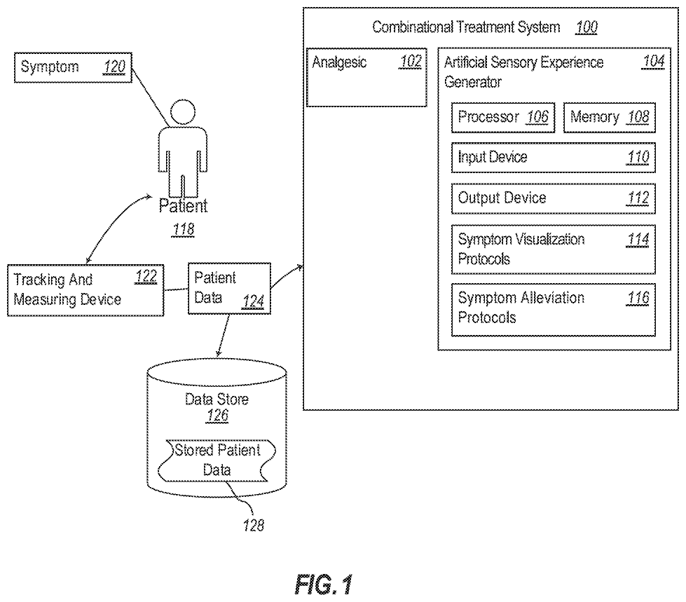

Referring now to FIG. 1, illustrated is an exemplary combinatorial therapeutic system 100 that combines administration of an analgesic and an artificial sensory experience to a patient. The combinatorial therapeutic system 100 includes an analgesic 102 and an artificial sensory experience generator 104.

The analgesic 102 includes a centrally and peripherally acting analgesic, preferably oliceridine and/or bupivacaine-meloxicam (and/or other analgesic drugs with similar mechanism of action and/or similar intended uses), with systemic exposure. The analgesic 102 can be administered to a patient 118 in a combinatorial treatment method in dosage forms, frequencies, and concentrations that are at most equal to the approved and/or prescribed dosage for a given patient, as known in the art. However, in some embodiments, the analgesic 102 can beneficially be administered to the patient 118 in a combinatorial treatment method in frequencies and/or concentrations less than the patient's baseline or prescribed dosage prior to administration of the combinatorial treatment method.

### Analgesics

As discussed herein, analgesics that can be used in the combinatorial therapeutic systems and methods described herein include those centrally and peripherally acting analgesics with systemic exposure. Preferred analgesics include oliceridine and bupivacaine-meloxicam.

In some embodiments, the centrally and peripherally acting analgesics with systemic exposure may additionally or alternatively include a different drug which has similar molecular structure, similar mechanism of action, and/or similar intended use as oliceridine or bupivacaine-meloxicam.

Although the present invention is primarily focused on combinatorial therapy involving patients suffering from chronic pain who are or might start using analgesics prescribed for pain, with one potential desired result being the avoidance of addiction or abuse of these drugs, it will be appreciated that combinatorial therapy involving artificial sensory experience can also be used on subjects already addicted to prescription drugs with the goal of reducing addiction and abuse, or on subjects with (severe) acute pain who are prescribed these analgesics in high doses or for extended periods of time.

### Artificial Sensory Experience

Any one or more of the foregoing analgesics 102 can be combined with an artificial sensory experience produced by the artificial sensory experience generator 104 to achieve at least some of the above-mentioned advantageous effects. In some embodiments, the artificial sensory experience generator 104 can produce, for example, visual and/or other sensory stimuli to allow a patient 118 to virtually experience their symptom 120. As provided herein, the symptom 120 includes the multi-factorial experience of pain, although other symptoms are within the scope of this disclosure. In a preferred embodiment, the artificial sensory experience generator 104 enables the patient 118 to virtually experience aspects of the symptom 120 in accordance with the patient's own experience of the symptom 120. Stated another way, the artificial sensory experience generator 104 can be configured to reproduce one or more stimuli associated with a symptom 120 in a patient- and/or symptom-specific manner such that the artificial sensory experience is an accurate virtual representation of one or more aspects of the patient's symptom.

Aspects of the artificial sensory experience can be based on, for example, information received from the user via an input device 110. The user input can be incorporated into one or more of a symptom visualization protocol 114 or symptom alleviation protocol 116, which when processed by the processor 106 of the artificial sensory experience generator 104, causes the output device 112 to produce sensory stimuli representative of the symptom 120.

The principles described herein are not limited to precise input or output devices, and the selection of input and output devices can depend on the nature of the artificial sensory experience administered to the patient. Nevertheless, exemplary input devices include microphones, touchscreens, motion tracking devices, projections, holograms, cameras, keyboards, stylus, mouse, hand-held controllers—such as hand-held virtual reality controllers, other pointer input devices, sensors of any type (e.g., motion, eye-tracking), and so forth. In a preferred embodiment, the input device includes a user interface for use in communicating information from/to a user. This includes, for example, a virtual control panel or similar.

The artificial sensory experience enabled by computing systems disclosed herein can include single or multi-sensory feedback and can be implemented by any number and/or type of output device associated with the computing system. For example, visual stimulus/feedback can be provided through a display, tactile feedback can be provided through a wearable housing a haptic element (e.g., haptic clothing like a haptic vest, haptic suit, and/or haptic gloves or a handheld device having a resonant actuator or the like), auditory feedback can be provided through speakers (e.g., standalone speakers, headphones, etc.), olfactory feedback can be provided by an olfactory feedback apparatus as known in the art or by a pre-selected set of defined scents or aromas, and gustatory feedback can be enabled by intra-oral devices known in the art and/or a pre-selected set of defined taste substances (e.g., spices, confections, chemicals, etc.).

As used herein, the term “displays” is meant to include devices that provide visual stimuli in the form of images, video, projections, holograms, or the like. Accordingly, a display can include a monitor or screen configured to produce images and/or video. A display can additionally include projectors configured to project images or video onto a surface and those configured for holography. A display can additionally include headsets or eyewear configured for virtual reality, augmented reality, and/or mixed reality. Unless otherwise specified, artificial sensory experience embodiments described herein can be implemented in any of a virtual reality, augmented reality, and/or mixed reality configuration.

The artificial sensory experience can, in some embodiments, mimic the patient's current pain level, or in some embodiments, the artificial sensory experience can mimic or represent a desired pain level, such that the effect of a systemic, centrally and peripherally acting analgesic is synergistically enhanced, and/or a lower dose of the systemic, centrally and peripherally acting analgesic can be administered with the same perceived therapeutic effect. In some embodiments, the artificial sensory experience can mimic the patient's current pain experience and then change the experience to represent the same pain as a neutral (e.g., an absence of pain) or even a pleasant experience. In some embodiments, the artificial sensory experience can mimic the effects the user might get from receiving the analgesic (e.g., momentary blurring of vision, dulling of senses) to, for example, give the illusion that (more of) the analgesic was taken.

In some embodiments, the artificial sensory experience generator 104 can produce, visual and/or other sensory stimuli to allow a patient to virtually experience their symptom. In some embodiments, the symptom can be based on input and customization from the user. For example, as discussed in more detail elsewhere herein, the user can “draw” a virtual representation of his/her symptom that is then displayed within the artificial sensory environment. The term “draw” as used herein refers to creating a virtual representation of the symptom in at least 1 dimension. For example, a “drawn” 1D visual representation can refer to a visual dot. This virtual representation can take on multiple dimensions (e.g., 2D and/or 3D objects in addition or alternative to 1D objects). The term “drawing” also encompasses other sensory effects and includes audio/aural, tactile, olfactory or gustatory aspects of the symptom. In some embodiments the symptom can be selected from a set of symptom templates; e.g., chronic lower back pain, fibromyalgia, sciatica, a migraine headache, or other common symptom forms or pain classifications. In some embodiments, the symptom is selected by a computer system or a helper (e.g., a human familiar with the system who can assist the user in selecting, customizing, or otherwise configuring the symptom, and/or a physician who has diagnosed the symptom and is familiar with its symptomology can configure the symptom). In some embodiments the symptom may be present (e.g., some combination of visible, audible, haptically active, etc.) for some aspects of the artificial sensory experience but not for others. In some embodiments, the system is configured to generate an artificial sensory experience that comprises multiple versions of the artificial sensory experience, some of which involve the symptom, while others do not.

As one example, the artificial sensory experience generator 104 generates a representation of the user's real-world symptom (or set of symptoms) by providing a shape (e.g., a sphere or other shape) located on or within the user (or an avatar representing the user) at a location associated with the real-world symptom(s) of the user (e.g., on or within the back of a user with back pain). The shape can be configured with features that match the user's experience of the symptom(s). For example, the shape may be configured with one or more of: a size that represents the perceived size or coverage of the symptom(s); a color that represents the perceived severity of the symptom(s) (e.g., brighter and/or more intense colors for worse symptoms); a movement pattern that represents the perceived severity of the symptom(s) (e.g., a “throbbing”/“pulsing” motion, movements that are synchronized with the throbbing or pulsing experienced by that user); or other visual features that represent the user's experience of the symptom(s). Other sensory stimuli may be additionally or alternatively generated by the artificial sensory experience generator 104 to represent the symptom. For example, audio may be associated with the user's symptom(s) and may be varied according to one or more of: volume (e.g., louder sounds associated with higher levels of the symptom(s)), type of sound (e.g., harsher sounds associated with higher levels of the symptom(s)), beat/rhythm of sound (e.g., to match a “throbbing” experienced by the user), or other auditory features that represent the user's experience of the symptom(s).

In some embodiments, the artificial sensory experience involves an alleviation protocol. In some embodiments this alleviation protocol may involve a change to the symptom that represents an improvement, whether physical or psychological, to the symptom. This may include, for example, a reduction or a removal of the symptom. For example, if the symptom is represented by a sphere or other shape located on or within the user (or an avatar representing the user) at a location associated with the real-world symptom(s) of the user (e.g., on or within the back of a user with back pain), the alleviation protocol can function to reduce the size of the shape, change the color of the shape (e.g., from a bright and/or intense color to a less bright, more bleached and/or more neutral color), change the movement of the shape (e.g., to a less frequent “throbbing” motion or movement away from the user or avatar), change the transparency of the shape, and/or otherwise change the visualization of the shape over time in a manner that suggests an alleviation in the symptom. Other sensory stimuli generated by the artificial sensory experience generator 104 can also be modified. For example, audio associated with the symptom may be modified through a reduction in volume, a change from harsher sounds/tones to milder sounds/tones, a change in beat frequency of “throbbing” sounds, and/or other auditory changes over time that suggest an alleviation of the symptom. In some embodiments, symptom alleviation may be represented by one or more of: a reduction in size of, an increase of a distance from, an evaporation of, a recoloring/discoloration of, a dilution of, a diffusion of, a dissipation of, a relocation of, a reduction in frequency of, a distortion of, a disappearing of, a washing or blowing away of, a removal of, a throwing away of, a silencing of, a slowing of, a melting of, a healing of, a stilling of, or a cooling of the symptom. In some embodiments, there is no alleviation protocol (or it may be an optional module/component). In some embodiments, the symptom stays the same. In some embodiments, the system 100 is configured to provide multiple versions of the artificial sensory experience, some of which involve a symptom alleviation protocol, while others do not.

In some embodiments, the virtual representation (comprising one or more visual, aural/audio, tactile, olfactory, and/or gustatory stimuli) can change over time to help the user reassess or reattribute the meaning of (aspects of) the symptom, thereby changing the overall (psychological) effect of the symptom; e.g., a user who drew a dark gray cloud to represent a dull-feeling chronic lower back pain may be able to redraw the cloud (or the system automatically redraws it) as the user begins to realize that some of those sensations are actually just tightness or tension and not pain.

In some embodiments, the artificial sensory environment includes audio, visual or other multimedia guidance within the artificial sensory environment to train or reinforce one or more symptom handling techniques for recognizing, understanding, or coping with the symptom, such as one or more of specific inputs, affirmations, or instructions from a helper or from the user regarding the user's ability to control, dampen, alleviate or eliminate the symptom, or turning of symptom into a neutral or positive experience.

In some embodiments, the artificial sensory experience includes a combination of tactile, auditory, visual, olfactory, and/or gustatory feedback.

For example, augmented reality can provide auditory and visual feedback at a smart phone that augments a portion of the patient's body—the portion experiencing pain, for example—to illustrate the patient's pain at the same level or a desired level of pain. This can help the user to visualize and hear the pain similar to how the user experiences the pain, and by doing so, begin to control the intensity and/or duration of the pain. In some examples this effect may be further enhanced by providing visual, auditory, tactile or other stimuli, such as blurriness of vision or tingling, the user might experience if they actually used more analgesic. The analgesic taken therewith can have increased efficacy as a result of the augmented reality feedback.

In some embodiments, the output device 112 can provide a virtual representation of the symptom 120 in any of a 2D/3D/4D digital system. It should be appreciated that the foregoing dimensionality of the digital systems is made with respect to a user's perspective of the digital system. For example, a 2D digital system can include visual representations provided on a flat display, such as a laptop or desktop monitor, the screen of a mobile electronic device, or similar.

These multi-dimensional digital systems can be coupled with other devices producing sensory stimuli (e.g., auditory stimulus provided by speakers, tactile stimulus provided by haptic clothing or devices, gustatory stimulus provided by intra-oral devices, and olfactory stimulus supplied by olfactory feedback apparatuses) to increase the dimensionality of the user experience. For example, the dimensionality of the aforementioned 2D digital systems can be increased by the addition of another visual dimension or by the addition of another sensory stimulus. With respect to the running example of a 2D digital system providing visual representations on a flat display, the dimensionality of the digital system can be increased to a 3D digital system through the incorporation of an additional visual dimension. For example, providing the visual representations in an immersive virtual reality experience provides three-dimensional visual stimulus to the user. As such, most virtual reality systems are at least 3D digital systems. It should be appreciated that some augmented reality and mixed reality implementations can also be considered 3D digital systems.

In some embodiments, a 3D digital system can include a 2D visual display having one or more additional, non-visual stimuli associated therewith. For example, a 3D digital system can be created by combining a 2D visual display implemented on a mobile electronic device with auditory stimulus provided by speakers associated with the mobile electronic device. The 2D visual display provides the first two dimensions of the eventual 3D digital system with the third dimension being provided by the additional sensory stimulus—the auditory stimulus provided by the speakers. Similarly, the addition of tactile, olfactory, or gustatory stimuli in place of the auditory stimulus would also create a 3D digital system.

Of note, as the dimensionality of artificial sensory experiences is defined herein, the type of visual stimulus forms the foundation of the dimensionality: a visual stimulus like that observed on the screen of a mobile electronic device is a 2D visual stimulus whereas a visual stimulus like that of a virtual reality environment is a 3D visual stimulus. An additional dimension can be added to the dimensionality of 2D or 3D visual stimuli to create a 3D or 4D artificial sensory experience. For example, a 2D visual stimulus can become a 3D artificial sensory experience by including an additional sensory stimulus, such as a tactile, aural, olfactory, or gustatory stimulus. In a similar fashion, a 3D visual display (e.g., virtual reality) can become a 4D artificial sensory experience through the layering of a non-visual sensory stimulus, such as an auditory stimulus provided by speakers, a tactile stimulus provided by haptic clothing or devices, a gustatory stimulus provided by intra-oral devices, and an olfactory stimulus supplied by olfactory feedback apparatuses.

However, the layering of additional different stimuli may not increase the dimensionality of the system. That is, a 3D artificial sensory experience made of a 2D visual stimulus and a tactile stimulus, for example, does not become a 4D artificial sensory experience with the addition of another sensory stimulus, and a 4D artificial sensory experience made of a 3D visual stimulus and a tactile stimulus, does not become a 5D artificial sensory experience with the addition of another sensory stimulus. Further illustrating this point, while the addition of a first additional stimulus—such as an auditory stimulus—to a 2D visual display would create a 3D artificial sensory experience, layering additional different stimuli in addition to the auditory stimulus and 2D visual display would still result in a 3D artificial sensory experience, as those terms are used herein.

With continued reference to FIG. 1, the artificial sensory experience generator 104 is configured to provide a sensory experience to a user. These artificial sensory experience generators and associated artificial sensory experiences can be implemented using any of an array of computer systems, which are now increasingly taking a wide variety of forms. “Artificial sensory experience generators,” as that term is used herein, include any computer system, device, or combination thereof that includes at least one processor and a physical and tangible computer-readable memory capable of having thereon computer-executable instructions that are executable by the processor. The breadth and operability of computer systems is discussed in greater detail below.

In short, computer systems can include a variety of devices, such as mobile phones, electronic appliances, laptop computers, tablet computers, wearable electronic devices, desktop computers, mainframes, and the like. As used in this disclosure, although not limited in this way, computer systems store and operate artificial sensory experience generators to enable the visualization and virtual alleviation of a patient's symptom (i.e., pain) as part of the combinatorial treatment systems disclosed herein and can be operably linked to various input and output devices. A computing system may be distributed over a network environment and may include multiple constituent computing systems (e.g., a cloud computing environment). In a cloud computing environment, program modules may be located in both local and remote memory storage devices.

In a preferred embodiment, the artificial sensory experience is virtual reality, more preferably virtual reality neuropsychological therapy. As used herein, the term “virtual reality neuropsychological therapy” or “VRNT” includes virtual reality systems for symptom management (e.g., pain management) that provide a patient with a virtual, yet realistic and strongly impactful, representation of the patient's symptoms (e.g., pain) and potentially even their alleviation, as disclosed in U.S. Pat. No. 10,249,391, which is incorporated herein by reference (which uses the term “virtual reality neuro-therapy”).

In another preferred embodiment, the artificial sensory experience is virtual reality distraction therapy or Virtual Reality relaxation therapy. These therapies utilize a virtual reality environment to distract the patient from their pain or to provide a therapeutic degree of relaxation and/or escapism. They can affect its therapeutic benefit by relaxing the patient and/or commanding the patient's focus away from the symptom (e.g., pain) to refocus the patient on a task such as collecting coins or shooting zombies in a video game simulated in the virtual environment. Research has shown that the capacity of humans to focus or pay attention is limited. An individual's focus or concentration on a symptomatic experience (e.g., pain) allows them to perceive the pain to a certain extent. Accordingly, perception of a symptomatic experience such as pain decreases when the individual's attention is distracted away from the stimulus, or more precisely, refocused on another task which provides high cognitive load.

While many of the results and discussion provided herein are directed to combinatorial therapies using VRNT and systemic, centrally and peripherally acting analgesics, it should be appreciated virtual reality distraction therapy in combination with systemic, centrally and peripherally acting analgesics is also expected to have unexpected combinatorial and/or synergistic benefits. More particularly, combinatorial therapies that include virtual reality distraction therapy and any of the one or more analgesics disclosed herein can have an unexpected synergistic therapeutic effect beyond that which could be achieved using either alone.

Further, in some embodiments, combinatorial treatment system and methods can include a systematic combination of VRNT and virtual reality distraction therapy with systemic, centrally and peripherally acting analgesics to provide the beneficial effects disclosed herein. For example, virtual reality distraction therapy can be used during heightened symptomatic periods in combination with systemic, centrally and peripherally acting analgesics to synergistically provide a therapeutic benefit to the patient, and VRNT can be used subsequently to assist the patient in reducing their reliance on the systemic, centrally and peripherally acting analgesic and/or to make the systemic, centrally and peripherally acting analgesic more effective at the same or lower concentrations. Other combinations of VRNT and virtual reality distraction therapy are also considered within the scope of this disclosure.

In some instances, a user's psychological state prevents the systemic, centrally and peripherally acting analgesic from working to its full efficacy. By coupling the systemic, centrally and peripherally acting analgesic with the artificial sensory experience, the user may be able to enter or change their psychological state that allows the systemic, centrally and peripherally acting analgesic to reach its full potency and/or increase its effectiveness beyond (e.g., in duration or strength) its historic utility with the given patient.

In a preferred embodiment, the artificial sensory experience is virtual reality. The patient can be immersed within the virtual reality using, for example, a head mounted display or other headgear (for visual feedback), headphones (for auditory feedback), and paddles or controllers (which may include haptic elements for tactile feedback systems). The virtual reality environment can, for example, instruct the user to accomplish a task. The task may include a meditative session to calm and/or relax the individual, a meditative session to visualize the isolation and/or elimination of the pain, drawing and/or visualizing the pain (as in VRNT) to illustrate the pain as it would visually appear, for example, in location, color, intensity, and description (e.g., aching, pulsing, throbbing, stabbing, shooting, cramping, gnawing, burning, etc.) on an avatar representative of the patient. The pain may further be associated with sounds, smells, and/or tactile feedback that help the patient become immersed in a visual representation of the pain. The computer system and/or the user can then interact with the visualized pain and/or visualize its dampening, control, elimination, or the turning of the pain into a neutral or positive experience. In some embodiments, as the systemic, centrally and peripherally acting analgesic takes effect (or as it is enabled to affect), a positive feedback loop is generated where the user's visualization of the pain is decreasing and the physiologic response to the systemic, centrally and peripherally acting analgesic is decreasing pain in the patient.

### Additional Embodiment of an Artificial Sensory Experience Generator

The following discloses one presently preferred embodiment of an artificial sensory experience generator 104. Although some specific features are described by way of representative example, it will be understood that the other alternatives and options disclosed herein may likewise be utilized. In this example, the system 100 includes a head mounted virtual reality display (which can in some alternatives be substituted with one or more of the other “displays” described herein) as one input device 110, processor 106, and associated memory 108 configured for storing data instructions.

In this embodiment, the artificial sensory experience generator 104 is a virtual reality device. In other implementations, the artificial sensory experience generator 104 is an augmented reality, mixed reality, or holographic device (any of such being optionally linked to a computer system). The artificial sensory experience comprises an avatar, representing the user (patient), within a virtual (or in some instances partly virtual) environment, such as a room, a space, a landscape, or an imaginary location/environment. At the beginning of the therapy a representation of the chronic pain is created on or in the avatar to represent the user's pain experience. This is done either by the user “drawing” 1D, 2D, and/or 3D objects representing their chronic pain, or the user selecting from a set of prefab pain drawings (e.g., 1D, 2D, and/or 3D objects which look like or represent aching, throbbing, sore, stabbing, shooting, stabbing, cramping, gnawing, splitting, heavy, or burning pain). Alternatively, the computer system can generate a “standard” representation of the chronic pain type being treated; e.g., chronic lower back pain, migraine headache, sciatica). As discussed elsewhere, a “drawing” does not just have to refer to visual aspects, but may include aural/audio, tactile, olfactory, and/or gustatory features.

In some implementations the pain “drawing” can be customized to better correspond to the user's own pain experience by, for example, changing one or more of the following: the location, shape, size, intensity, color, or other feature of the drawing. In some implementations, the pain representation may, in addition to the visual component, include one or more of the following: an aural or audio component, a tactile component, an olfactory component, and/or a gustatory component. Customizing these aspects could then, for example, involve changing the volume, tone, intensity, flavor, smell, or taste.

After a symptom/pain representation has been selected, the user begins using the artificial sensory experience generator 104. The artificial sensory experience generator 104 provides multiple experiences or “modules”. Each module may have a slightly different purpose; e.g., creating a slightly different experience. Most of the modules will involve audio, visual, or other multi-dimensional guidance; e.g., a voiceover may guide the user to have a certain interaction with the avatar, the pain representation, or the pain itself to help the user evaluate the pain symptoms or to experience the pain symptoms differently. In some modules the user may see the pain symptoms on or within the avatar, while in others the avatar may not show the pain symptoms. In some modules the pain symptoms will change over time, for example representing an improvement of the pain.

In some modules the user will also experience visual, audio, tactile, olfactory and/or gustatory changes in the artificial sensory environment which will “feel” like the experience of taking the systemic, centrally, and peripherally acting analgesic. For example, the user may take a dose of the analgesic, and then run a module in which they experience changes in focus, or color or a shakiness in images similar to the euphoric rush of taking the analgesic. At the same time, a voiceover guides the user through techniques for gaining greater control over the psychological components of pain. This may lead the user to experience greater than normal analgesic effect, and a greater sense of control over the psychological effects of pain.

The combinatorial treatment system may be used over a period of time (e.g., 8-12 weeks) to treat chronic pain.

In other versions of the preferred embodiment, the memory 108 may be configured for storing data instructions that, when executed by the processor 106, cause the system to perform a method for representing symptoms (and optionally also representing symptom alleviation). In some embodiments, the system 100 enables the act of creating, by the processor 106, an artificial sensory experience that includes an avatar of a user, wherein the avatar represents a body of the user. The artificial sensory experience may also include a user interface enabling the user to describe a symptom or to select from a set of predefined descriptions (i.e., “prefabs”). The artificial sensory experience may include a landscape, a scene, or a room, for example. The system 100 further enables a head mounted display (or alternatively, one of the other displays described herein) to generate the artificial sensory experience.

The system 100 further enables a symptom visualization protocol 114 by receiving a description of a symptom (e.g., pain and/or chronic pain) via the user interface. The description may be for various pain types including one or more of aching, throbbing, sore, stabbing, shooting, cramping, gnawing, splitting, heavy, or burning. In some implementations, the system 100 further enables the user to select from the various pain types and then modify the pain's location, size, intensity, frequency, depth, or saturation. This enables the user to customize the representation of the symptom to the user's own experience of the symptom. Alternatively, the system 100 receives a selected predefined description via the user interface. The system 100 further causes the head mounted display to generate the symptom representation within the artificial sensory experience (e.g., by locating the symptom representation on or within the avatar of the user).

The system 100 further enables an alleviation protocol 116 by causing at least a portion of the symptom representation to change continuously over time to human perception to, for example, reduce or eliminate the symptom representation. This can correspond to one or more of: a reduction in size of, an increase of a distance from, an evaporation of, a recoloring/discoloration of, a dilution of, a diffusion of, a dissipation of, a relocation of, a reduction in frequency of, a distortion of, a disappearing of, a washing or blowing away of, a removal of, a throwing away of, a silencing of, a slowing of, a melting of, a healing of, a stilling of, or a cooling of the symptom.

In some implementations, the system 100 also provides audiovisual or other multimedia guidance to accompany the symptom visualization protocol 114 and/or symptom alleviation protocol 116. Examples of such audiovisual or other multimedia guidance include voiceovers to guide the user during interactions with the artificial sensory environment, the avatar, the pain representation, or the pain itself. The audiovisual or other multimedia guidance can thereby guide the user through techniques for gaining greater control over the psychological components of pain.

### Computer Systems of the Present Disclosure

As provided above, artificial sensory experience generators can include computer systems. In some embodiments, these computer systems are configured to, for example, execute symptom visualization and/or alleviation protocols, receive input from a user, and output various stimuli to the user via one or more output devices (such as those disclosed herein). It will be appreciated that computer systems are increasingly taking a wide variety of forms. In this description and in the claims, the term “computer system” or “computing system” is defined broadly as including any device or system—or combination thereof—that includes at least one physical and tangible processor and a physical and tangible memory capable of having thereon computer-executable instructions that may be executed by a processor. By way of example, not limitation, the term “computer system” or “computing system,” as used herein is intended to include personal computers, desktop computers, laptop computers, tablets, mobile electronic devices (e.g., smart phones and tablets), microprocessor-based or programmable consumer electronics, minicomputers, mainframe computers, multi-processor systems, network PCs, distributed computing systems, datacenters, message processors, routers, switches, and even devices that conventionally have not been considered a computing system, such as standalone mobile virtual reality headsets or wearables (e.g., glasses, smart watches, personal health monitors).

Within a computing system, the associated memory may take any form and may depend on the nature and form of the computing system. The memory can be physical system memory, which includes volatile memory, non-volatile memory, or some combination of the two. The term “memory” may also be used herein to refer to non-volatile mass storage such as physical storage media.

The computing system also has thereon multiple structures often referred to as an “executable component.” For instance, the memory of a computing system can include an executable component. The term “executable component” is the name for a structure that is well understood to one of ordinary skill in the art in the field of computing as being a structure that can be software, hardware, or a combination thereof.

For instance, when implemented in software, one of ordinary skill in the art would understand that the structure of an executable component may include software objects, routines, methods, and so forth, that may be executed by one or more processors on the computing system, whether such an executable component exists in the heap of a computing system, or whether the executable component exists on computer-readable storage media. The structure of the executable component exists on a computer-readable medium in such a form that it is operable, when executed by one or more processors of the computing system, to cause the computing system to perform one or more functions, such as the functions and methods described herein. As a non-limiting example germane to the present application, an “executable component” can include computer-executable instructions for instantiating an artificial sensory experience. Such a structure may be computer-readable directly by a processor—as is the case if the executable component were binary. Alternatively, the structure may be structured to be interpretable and/or compiled—whether in a single stage or in multiple stages—so as to generate such binary that is directly interpretable by a processor.

The term “executable component” is also well understood by one of ordinary skill as including structures that are implemented exclusively or near-exclusively in hardware logic components, such as within a field programmable gate array (FPGA), an application specific integrated circuit (ASIC), Program-specific Standard Products (ASSPs), System-on-a-chip systems (SOCs), Complex Programmable Logic Devices (CPLDs), or any other specialized circuit. Accordingly, the term “executable component” is a term for a structure that is well understood by those of ordinary skill in the art of computing, whether implemented in software, hardware, or a combination thereof.

The terms “component,” “service,” “engine,” “module,” “control,” “generator,” or the like may also be used in this description. As used in this description and in this case, these terms—whether expressed with or without a modifying clause—are also intended to be synonymous with the term “executable component” and thus also have a structure that is well understood by those of ordinary skill in the art of computing. As a non-limiting example, the artificial sensory experience generators disclosed herein can include symptom visualization and alleviation protocols stored within memory (e.g., non-volatile memory) as computer-executable instructions that, when executed by the one or more processors (e.g., hardware processors) of the artificial sensory experience generator, cause the computer system that is (or includes) the artificial sensory experience generator to execute an artificial sensory experience, such as VRNT or virtual reality distraction therapy.

Accordingly, embodiments described herein may comprise or utilize a special purpose or general-purpose computing system. Embodiments described herein also include physical and other computer-readable media for carrying or storing computer-executable instructions and/or data structures. Such computer-readable media can be any available media that can be accessed by a general purpose or special purpose computing system. Computer-readable media that store computer-executable instructions are physical storage media. Computer-readable media that carry computer-executable instructions are transmission media. Thus, by way of example—not limitation—embodiments disclosed or envisioned herein can comprise at least two distinctly different kinds of computer-readable media: storage media and transmission media.

Computer-readable storage media include RAM, ROM, EEPROM, solid state drives (“SSDs”), flash memory, phase-change memory (“PCM”), CD-ROM or other optical disk storage, magnetic disk storage or other magnetic storage devices, or any other physical and tangible storage medium that can be used to store desired program code in the form of computer-executable instructions or data structures and that can be accessed and executed by a general purpose or special purpose computing system to implement the disclosed functionality of the invention. For example, computer-executable instructions may be embodied on one or more computer-readable storage media to form a computer program product.

Transmission media can include a network and/or data links that can be used to carry desired program code in the form of computer-executable instructions or data structures and that can be accessed and executed by a general purpose or special purpose computing system. Combinations of the above should also be included within the scope of computer-readable media.

Further, upon reaching various computing system components, program code in the form of computer-executable instructions or data structures can be transferred automatically from transmission media to storage media (or vice versa). For example, computer-executable instructions or data structures received over a network or data link can be buffered in RAM within a network interface module (e.g., a “NIC”) and then eventually transferred to computing system RAM and/or to less volatile storage media at a computing system. Thus, it should be understood that storage media can be included in computing system components that also—or even primarily—utilize transmission media.

Those skilled in the art will further appreciate that a computing system (e.g., an artificial sensory experience generator that is or includes a computing system) may also contain communication channels that allow the computing system to communicate with other computing systems over, for example, a network. Accordingly, the methods described herein may be practiced in network computing environments with many types of computing systems and computing system configurations. The disclosed methods may also be practiced in distributed system environments where local and/or remote computing systems, which are linked through a network (either by hardwired data links, wireless data links, or by a combination of hardwired and wireless data links), both perform tasks. In a distributed system environment, the processing, memory, and/or storage capability may be distributed as well.

As a non-limiting example, symptom visualization protocols described herein may be stored on a network server where they are accessed and executed in real time by an end-user device, such as a smart phone or network connected smart display (e.g., XR glasses). Additionally, or alternatively, end-user data can be transmitted to a network server, and symptom visualization protocols can be updated based on the received end-user data to reflect the user's experience more accurately. In some embodiments, this may be a computationally intensive process, and by processing these data over a system of network-connected computing systems, the overall processing time can be significantly reduced while simultaneously reducing local processing demands on the visualization-protocol-hosting server.

Those skilled in the art will also appreciate that the disclosed methods may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

Although the subject matter described herein is provided in language specific to structural features and/or methodological acts, it is to be understood that the subject matter defined in the appended claims is not necessarily limited to the described features or acts so described. Rather, the described features and acts are disclosed as example forms of implementing the claims.

### EXAMPLES

The following prophetic examples illustrate how combinatorial therapy involving administration of analgesic coupled with artificial sensory experiences to patients with (chronic) pain can greatly improve one or more favorable outcomes. While the examples are hypothetical in nature, they are based on qualitative observations involving VRNT administered to and received by patients on the one hand, and analgesia administered to and taken by patients on the other. Based on extrapolations of such data, the inventors have concluded that combinatorial therapy involving administration of VRNT and analgesia will provide synergistic effects and unexpectedly favorable outcomes in several areas.

Artificial sensory experience (e.g., VRNT) and analgesia have fundamentally different modes of action. VRNT and other artificial sensory experiences are sensory and psychological in nature, with the patient receiving primarily visual stimulation and secondarily/optionally auditory, tactile, olfactory, or gustatory stimulation. These are believed to affect the patient's conscious and/or subconscious pain experience, understanding and interpretation of pain; however, they are not known to directly affect biological causes of “pain” (or danger) signals from the body (signals from the nervous system which the brain interprets as pain)—such as injury, tissue damage, nerve damage or disease—or the nerves (nociceptor neurons) that respond to damaging or potentially damaging stimuli and transmit these signals via the spine to the brain (where the signals are interpreted as pain or something else).

Analgesics including systemic, centrally and peripherally acting analgesics, preferably oliceridine or bupivacaine-meloxicam with systemic exposure, are primarily physiological and biochemical in nature by blocking or interfering with transmission of pain impulses. They are secondarily psychological in the sense that they provide temporary relief of pain, which is positive and beneficial psychological experience. However, they can have serious negative psychological effects, such as minor or major intoxication and, worse, long-term addiction and withdrawal if reduced or curtailed.

It has now been found, or postulated, that combining the primarily psychological effects of artificial sensory experience, such as VRNT, with the primarily physiological effects of—specifically—systemically effected, centrally and peripherally acting analgesics can synergistically reinforce each other and yield unexpectedly beneficial and favorable outcomes. In some cases, VRNT or other artificial sensory experiences can substantially increase the physiological and psychological efficacy of analgesics in reducing pain. In other cases, systemic, centrally and peripherally acting analgesics can substantially increase the psychological and physiological efficacy of VRNT or other artificial sensory experiences in reducing the perception of pain and better understanding the nature of pain, such as making pain more benign or controllable in the mind of the patient.

While the description of combinatorial therapies focuses on patients' experience of pain, it will be appreciated that what constitutes “pain” can be manifest, experienced, or understood in different ways by different patients and/or by the same patient at different times. Examples of ways to understand the mean of pain include pain intensity, frequency of (severe) pain episodes, severity of pain episodes, secondary pain effects, such as emotional suffering, stress, depression, fear or anxiety, quality of life, mobility, movement, activity limitations, disability (level of), kinesiophobia (fear of movement), areas of body in pain, cessation of expansion of pain or deterioration of pain symptoms, predictability of pain, sense of “normal” body sensations, sense of self-efficacy (or sense of helplessness), overall satisfaction with treatment(s), life orientation, pain catastrophizing, anger/emotion regulation, pain attitude, pain coping, avoidance of surgery, and the like. Each of these can be used as outcome measures to measure the effectiveness of a therapy.

Favorable outcomes that can be provided by combinatorial therapy as disclosed herein include improved efficacy of systemic, centrally and peripherally acting analgesics as measured by one or more of: (1) an increase in the number of patients responding favorably to systemic, centrally and peripherally acting analgesics or responding to a greater extent to treatment; (2) a reduction in the amount of time required for systemic, centrally and peripherally acting analgesics to take effect; (3) a reduction in the dosage or amount of systemic, centrally and peripherally acting analgesics required for a given reduction in pain; (4) a reduction in the frequency of administration of systemic, centrally and peripherally acting analgesics to achieve a given reduction in pain; (5) an increase in duration of systemic, centrally and peripherally acting analgesic effect for a given dose of systemic, centrally and peripherally acting analgesic; (6) cumulative or lasting reduction of pain; and (7) an increase in the overall perceived efficacy of systemic, centrally and peripherally acting analgesic, such as a reduction in pain intensity experienced by the patient or other subjective or objective measures of efficacy disclosed herein.

Although favorable outcomes might be understood as a quantifiable reduction in the amount of systemic, centrally and peripherally acting analgesic required to achieve a given pain reduction and/or a quantifiable reduction in pain for a given amount of systemic, centrally and peripherally acting analgesic received by the patient, other favorable outcomes can involve improvements in the effectiveness of VRNT or other artificial sensory experiences, such as in their ability to permit the patient to be less emotionally affected by the pain or to reinterpret the pain to lessen the perception of severity even if the actual level of pain intensity remains the same.

It should be appreciated that favorable outcomes can be more subjective in nature and/or more generally defined. For example, the patient may not perceive a decrease in the intensity or duration of pain following combinatorial therapy but may nonetheless report a better quality of life or an improved ability to cope with or frame the pain such that the patient is less affected or impacted by the pain and better suited or able to engage in routine or desired activities. Favorable outcomes could additionally include increased optimism (or an increase in positive outlook on life) or an improvement in the patient's mood (or a decrease in negative emotions) that cannot be accounted for by administration of analgesic or artificial sensory experience alone.

Therefore, favorable outcomes that can be provided by combinatorial therapy as disclosed herein include improved efficacy of VRNT or other artificial sensory experience as measured by one or more of: (1) an increase in the number of patients responding favorably to VRNT or other artificial sensory experience or responding to a greater extent to VRNT or artificial sensory experience; (2) a reduction in the number of therapy sessions before initial beneficial effects and/or cumulative effects are experienced; (3) shorter session durations required to yield desired or meaningful results; (4) a lower frequency of sessions needed to reach desired or meaningful results; (5) an increase in the duration of the beneficial effects of VRNT or other artificial sensory experience; (6) cumulative or lasting reduction of pain; and (7) an increase in the effectiveness of VRNT or other artificial sensory experience, such as a reduction in pain intensity experienced by the patient or other measures of effective disclosed herein.

### Clinical Study 1: Combinatorial Treatment vs Analgesia Control

A group of 50 patients suffering from pre-existing chronic pain, some of whom have already begun taking analgesics such as systemic, centrally and peripherally acting analgesics to manage their pain, are given systemic, centrally and peripherally acting analgesics alone over a defined period of time to provide a baseline analgesia control data. After the analgesia control period, the patients are given combinatorial therapy, including artificial sensory experience (e.g., VNRT) and analgesics, over a defined period of time and the results are tabulated. During both the analgesia control period and the combinatorial therapy period, the following results over time are obtained by periodically surveying the patients and tabulated: (i) whether and how much the patients are responding to the analgesic, (ii) time between taking analgesic and onset of pain reduction over time, (iii) dosage of analgesic, (iv) frequency of administration of analgesic, (v) duration of analgesic effect for a given dose, (vi) cumulative or lasting reduction of pain; and (vii) overall effectiveness of analgesia, such as reduction in pain intensity, decrease in negative emotions, increase in positive outlook on life, and other measures.

### Example 1—Analgesia Control

The 50 patients with chronic pain are prescribed and administered an analgesic designed to reduce chronic pain for 30 days, which is the analgesia control period. With the understanding that the patients likely have different conditions causing chronic pain, different pain levels, are given different types and/or dosages of analgesics, and have different physiologies, psychologies, ages, and external circumstances, the experience of each patient taking an analgesic is nonetheless the baseline for that patient. Measures of improvement include a reduction of pain and/or analgesic usage while some involve an increase in efficacy of the analgesia and increased overall well-being of the patient. For purposes of quantifying the results and measuring the cumulative experience of all patients without changes in numeric scores of different metrics offsetting each other, a higher score for each metric will correlate with a more negative outcome and a lower score will correlate with a more positive outcome. Based on that assumption, on a scale of 1 to 10, with 1 being the best outcome and 10 being the worst outcome, a baseline score of 7 is assigned to each patient for each of the seven metrics, which means the cumulative baseline score for all 50 patients is 350 for each of the seven metrics, as shown in Table 1.

TABLE 1

| | Cumulative |
|---|---|
| | Baseline Score |
| | For Analgesia |
| Metric | Control |
| Patients responding to analgesics and/or extent | 350 |
| of patient response to analgesics | |
| Amount of time required for analgesic to take | 350 |
| effect | |
| Dosage or amount of analgesic required | 350 |
| for a given pain reduction | |
| Frequency of administration of analgesic to | 350 |
| achieve a given reduction in pain | |
| Duration of analgesic effect for a given dose | 350 |
| of analgesic | |
| Cumulative or lasting reduction of pain | 350 |
| Overall perceived efficacy of analgesic (e.g., | 350 |
| reduction in pain intensity or other subjective | |
| or objective measures of efficacy) | |

### Example 2—Combinatorial Therapy of VRNT and Analgesia

The same 50 patients in the analgesia control are administered combinatorial therapy involving artificial sensory experience (e.g., VRNT) and the systemic, centrally and peripherally acting analgesic prescribed and received by the patients during the analgesia control period. Although the patients likely have different conditions causing chronic pain, different pain levels, and have different physiologies, psychologies, ages, and external circumstances, the subjective experience of each patient during the combinatorial therapy period is compared to that same patient's experience during the analgesia control period and given a score for each of the seven metrics. Any improvement by a patient for a given metric can be quantified by a score between 1-6, with 6 being the smallest improvement and 1 being the greatest improvement. A score of 7 means no improvement, and a score between 8-10 means a worse outcome. The scores for all 50 of the patients in the study are added together to give a cumulative score, as shown in Table 2, which provides a statistically relevant comparison of the group experience as a whole during the combinatorial therapy period compared to the analgesia control period. While combinatorial therapy is not expected to provide a worse outcome than analgesia alone, there may be outliers who experience more pain or other worse outcome due to external circumstances (e.g., deteriorating health, other injury, sickness, emotionally traumatic event, negative reactions to selected VRNT regimen, and the like).

TABLE 2

| | Cumulative |
|---|---|
| | Combinatorial |
| Metric | Therapy Score |
| Patients responding to—and/or patient response to— | 280 |
| systemic, centrally and peripherally acting analgesics | |
| Amount of time required for systemic, centrally and | 230 |
| peripherally acting analgesic to take effect | |
| Dosage or amount of systemic, centrally and | 190 |
| peripherally acting analgesic required for a | |
| given pain reduction | |
| Frequency of administration of systemic, centrally | 220 |
| and peripherally acting analgesic to achieve | |
| a given reduction in pain | |
| Duration of systemic, centrally and peripherally | 150 |
| acting analgesic effect for a given dose of systemic, | |
| centrally and peripherally acting analgesic | |
| Cumulative or lasting reduction of pain | 200 |
| Overall perceived efficacy of systemic, centrally and | 190 |
| peripherally acting analgesic (e.g., reduction in pain | |
| intensity or other subjective or objective measures of | |
| efficacy) | |

As shown by the cumulative scores in Table 2, combinatorial therapy provides a net increase in positive outcomes for the group of 50 patients as a whole. Even if a subset of patients experience either no benefit or a worse outcome in one or more metrics, the cumulative scores show that the group as a whole experienced a net increase in beneficial outcomes in each metric or category when given combinatorial therapy compared to analgesia alone.

### Clinical Study 2: Combinatorial Treatment vs VRNT Control

A different group of 50 patients suffering from pre-existing chronic pain, some of whom have already begun using artificial sensory experience (VRNT) or analgesics to manage their pain, are provided with VRNT alone over a defined period of time to provide baseline VRNT control data. After the VRNT control period, the patients are given combinatorial therapy, including VNRT and systemic, centrally and peripherally acting analgesics, over a defined period of time and the results are tabulated. During both the VRNT control period and the combinatorial therapy period, the following results over time are obtained by periodically surveying the patients and tabulated: (i) whether and how much the patients are responding to VRNT, (ii) number of VRNT sessions before initial beneficial effects and/or cumulative effects are experienced, (iii) duration of VRNT sessions required to yield desired or meaningful results, (iv) frequency of VRNT sessions needed to reach desired or meaningful results, (v) duration of beneficial effects following VRNT sessions, (vi) cumulative or lasting reduction of pain; and (vii) overall effectiveness of VRNT, such as reduction in pain intensity, decrease in negative emotions, increase in positive outlook on life, and other measures.

### Example 3—VRNT Control

The 50 patients with chronic pain are prescribed and administered a VRNT regimen to address or mitigate chronic pain for 30 days, which is the VRNT control period. With the understanding that the patients likely have different conditions causing chronic pain, different pain levels, might respond differently to VRNT, and have different physiologies, psychologies, ages, and external circumstances, the experience of each patient receiving VRNT is nonetheless the baseline for that patient. Measures of improvement include a reduction of pain and/or number of VRNT sessions and/or session duration while some involve an increase in efficacy of VRNT and increased overall well-being of the patient. For purposes of quantifying the results and measuring the cumulative experience of all patients without changes in numeric scores of different metrics offsetting each other, a higher score for each metric will correlate with a more negative outcome and a lower score will correlate with a more positive outcome. Based on that assumption, on a scale of 1 to 10, with 1 being the best outcome and 10 being the worst outcome, a baseline score of 7 is assigned to each patient for each of the seven metrics, which means the cumulative baseline score for all 50 patients is 350 for each of the seven metrics, as shown in Table 3.

TABLE 3

| | Cumulative |
|---|---|
| | Baseline |
| | Score |
| | For VRNT |
| Metric | Control |
| Patients responding to VRNT and/or extent of | 350 |
| patient response to VRNT | |
| Number of sessions of VRNT before initial | 350 |
| beneficial or cumulative effects of VRNT | |
| are experienced | |
| Duration of VRNT sessions required for a given | 350 |
| reduction in pain or other beneficial result | |
| Frequency of VRNT sessions needed to reach a | 350 |
| given reduction in pain or other beneficial result | |
| Duration of beneficial effects following VRNT | 350 |
| sessions | |
| Cumulative or lasting reduction of pain | 350 |
| Overall perceived efficacy of VRNT (e.g., | 350 |
| reduction in pain intensity or other subjective | |
| or objective measures of efficacy) | |

### Example 4—Combinatorial Therapy of VRNT and Analgesia

The same 50 patients in the VRNT control are administered combinatorial therapy involving VRNT as received during the VRNT control period and a systemic, centrally and peripherally acting analgesic selected from oliceridine or bupivacaine-meloxicam. Although the patients likely have different conditions causing chronic pain, different pain levels, might respond differently to VRNT, and have different physiologies, psychologies, ages, and external circumstances, the subjective experience of each patient during the combinatorial therapy period is compared to that same patient's experience during the VRNT control period and given a score for each of the seven metrics. Any improvement by a patient for a given metric can be quantified by a score between 1-6, with 6 being the smallest improvement and 1 being the greatest improvement. A score of 7 means no improvement, and a score between 8-10 means a worse outcome. The scores for all 50 of the patients in the study are added together to give a cumulative score, as shown in Table 4, which provides a statistically relevant comparison of the group experience as a whole during the combinatorial therapy period compared to the VRNT control period. While combinatorial therapy is not expected to provide a worse outcome than analgesia alone, there may be outliers who experience more pain or other worse outcome due to external circumstances (e.g., deteriorating health, other injury, sickness, emotionally traumatic event, negative reactions to the selected systemic, centrally and peripherally acting analgesic, and the like).

TABLE 4

| | Cumulative |
|---|---|
| | Combinatorial |
| Metric | Therapy Score |
| Patients responding to VRNT and/or extent | 260 |
| of patient response to VRNT | |
| Number of sessions of VRNT before initial | 230 |
| beneficial or cumulative effects of VRNT | |
| are experienced | |
| Duration of VRNT sessions required for a given | 120 |
| reduction in pain or other beneficial result | |
| Frequency of VRNT sessions needed to reach a | 140 |
| given reduction in pain or other beneficial result | |
| Duration of beneficial effects following VRNT | 100 |
| sessions | |
| Cumulative or lasting reduction of pain | 130 |
| Overall perceived efficacy of VRNT (e.g., | 190 |
| reduction in pain intensity or other subjective | |
| or objective measures of efficacy) | |

As shown by the cumulative scores in Table 4, combinatorial therapy provides a net increase in positive outcomes for the group of 50 patients as a whole. Even if a subset of patients experience either no benefit or a worse outcome in one or more metrics, the cumulative scores show that the group as a whole experienced a net increase in beneficial outcomes in each metric or category when given combinatorial therapy compared to VRNT alone.

### Example 5

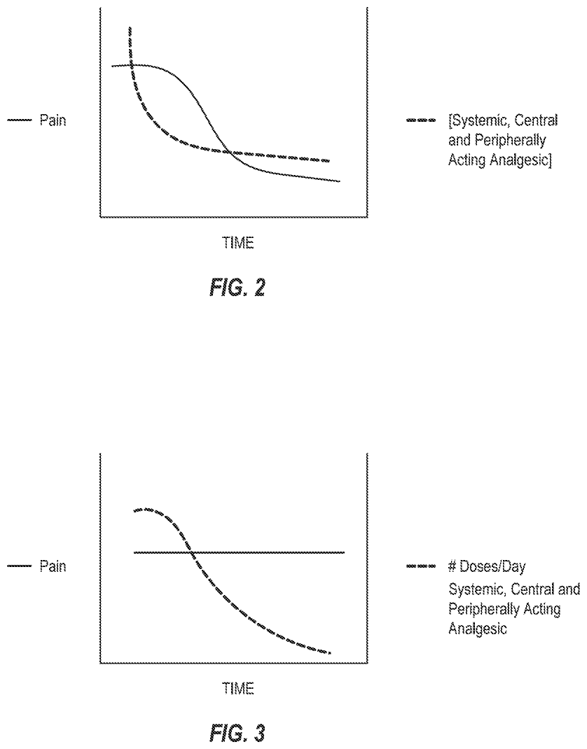

Referring now to FIG. 2, which is a graph illustrating an amount or perceived amount of pain over time (represented by the solid line) and a concentration of systemic, centrally and peripherally acting analgesic (represented as the broken line) over time that is used in a combinatorial therapeutic system/method in accordance with the present disclosure. As shown in the exemplary graph, one or both of the (perceived) pain level and systemic, centrally and peripherally acting analgesic concentration decrease over time. At the first point in time on the graph, the patient may be at a prescribed (e.g., clinically relevant) level of systemic, centrally and peripherally acting analgesic that has reached an equilibrium or steady state level of pain such that the systemic, centrally and peripherally acting analgesic is having no better pain-relieving effects by itself. However, when coupled with an artificial sensory experience (e.g., VRNT), the administered concentration of systemic, centrally and peripherally acting analgesic rapidly decreases to a lesser concentration. The reduced concentration of systemic, centrally and peripherally acting analgesic maintains the same level of (perceived or actual) pain in the patient at first, followed by a decrease in the patient's (perceived or actual) pain. In some embodiments, there is a delayed reduction of pain (as shown), but it should be appreciated that in some embodiments, the systemic, centrally and peripherally acting analgesic concentration can remain constant with a drop in the level of (perceived or actual) pain. Accordingly, as shown in FIG. 1, a combinatorial therapeutic system/method including a systemic, centrally and peripherally acting analgesic and artificial sensory experience can demonstrate a synergistically beneficial relationship.

It should be appreciated that although FIG. 2 illustrates the concentration of systemic, centrally and peripherally acting analgesic as decreasing rapidly to a second steady state concentration, other reaction modalities are possible. For example, the systemic, centrally and peripherally acting analgesic concentration may decrease linearly over time or may have multiple asymptotic thresholds. Similarly, the amount of perceived pain may have a different distribution or affect in response to the combinatorial therapy. The pain may decrease and increase cyclically with or without a trend of decreasing pain over time. The pain may decrease only insofar as the combinatorial therapy is being received or it may decrease in correlation with the amount of time (per session or longitudinally) the combinatorial therapy is administered. Regardless, the combinatorial therapy of a systemic, centrally and peripherally acting and an artificial sensory experience such as VRNT results in greater efficacy of the systemic, centrally and peripherally acting analgesic, greater efficacy of VRNT, and/or reduces the amount of pain experienced by the patient at a same level of systemic, centrally and peripherally acting analgesic and/or amount of VRNT.

### Example 6

As shown in FIG. 3, if the amount of pain (represented by the solid line of FIG. 3) is maintained (e.g., at or below a comfortable or acceptable level), the synergistic relation displayed by combinatorial therapeutic systems/methods disclosed herein can enable a decrease in the number of doses of systemic, centrally and peripherally acting analgesic taken per day and/or amount of VRNT required. It will be appreciated that systemic, centrally and peripherally acting analgesic usage can also be represented as a measure of the amount of daily equivalent doses of systemic, centrally and peripherally acting analgesic (represented by the broken line of FIG. 3). Similarly, the dosage amount can decrease in some embodiments. Additionally, or alternatively, the number of doses per day of systemic, centrally and peripherally acting analgesic can remain constant with a concomitant decrease in the amount of pain (not shown) and/or concomitant decrease in VRNT.

### Examples 7 & 8

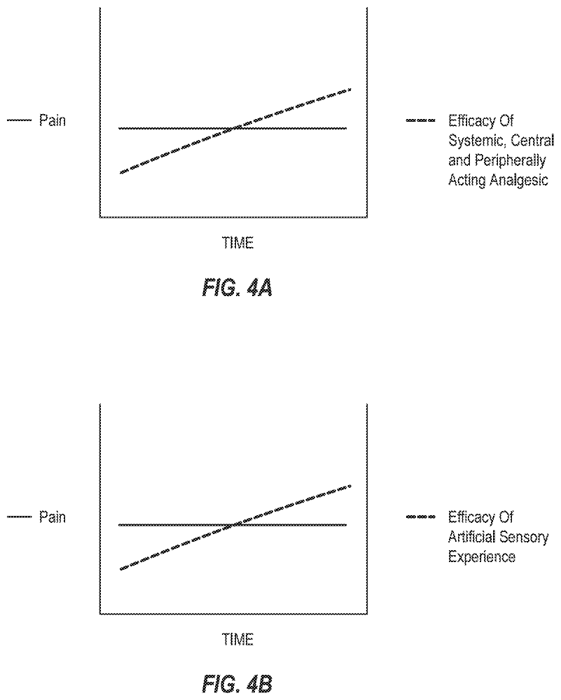

As shown in FIG. 4A, combinatorial therapeutic systems/methods disclosed herein can increase the efficacy of the systemic, centrally and peripherally acting analgesic over time while maintaining (or in some instances reducing) the amount of pain experienced by the patient. As shown in FIG. 4B, combinatorial therapeutic systems/methods disclosed herein can increase the efficacy of the artificial sensory experience (e.g., VRNT) over time while maintaining (or in some instances reducing) the amount of pain experienced by the patient. It should be appreciated that the synergistic benefits of combining an artificial sensory experience with a systemic, centrally and peripherally acting analgesic and described throughout the present disclosure, can cause an increase in the efficacy of the systemic, centrally and peripherally acting analgesic (FIG. 4A) and/or of VRNT (FIG. 4B), and this increase in efficacy can result in a reduced prescription concentration (e.g., FIG. 2) or number of doses (e.g., FIG. 3) of the systemic, centrally and peripherally acting analgesic. In some instances, it may additionally reduce the amount and/or number of times the artificial sensory experience is engaged by the user.

### Example 9

In one embodiment, the combinatorial system may be combined with an analgesic mechanism (e.g., drug drip, etc.), which controls the amount of systemic, centrally and peripherally acting analgesic being delivered to the subject. Measuring the amount of systemic, centrally and peripherally acting analgesic the subject uses may be used to break or reduce addictions or otherwise limit the total amount of systemic, centrally and peripherally acting analgesics received by and/or available to the population at large. In still another embodiment, the system may be combined with one or more biofeedback devices (e.g., heart rate/heart rate variability monitor, galvanic skin response monitor, electroencephalogram/EEG, fMRI, etc.) which may, for example, provide other data to evaluate patient pain levels or other patient data.

### Examples 10-12

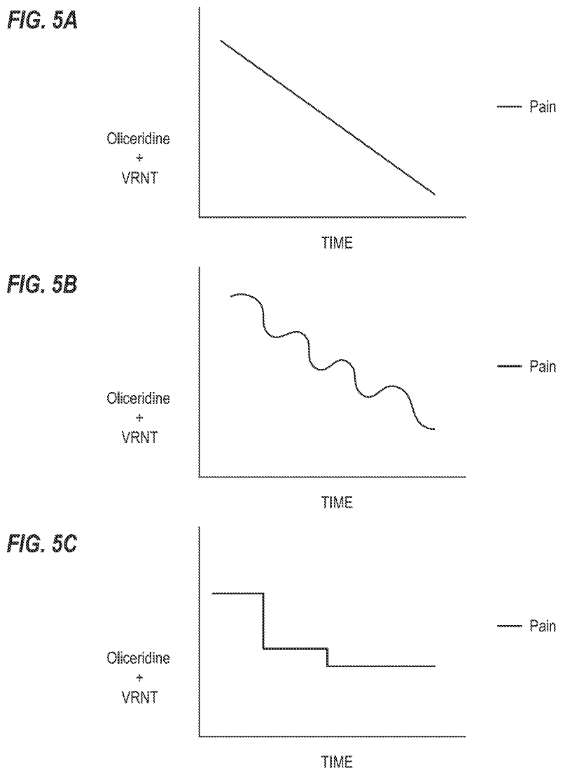

The synergistic effects of the disclosed combinatorial therapeutic systems/methods can be exemplified in a plurality of responses. For example, FIGS. 5A-5C illustrate a combinatorial therapy using oliceridine (as the systemic, centrally and peripherally acting analgesic) and VRNT (as the artificial sensory experience) with different potential patient responses. As many, if not all, patients may experience the synergistic benefits afforded by the disclosed combinatorial therapeutic systems/methods as an average benefit, the individual-specific responses may be different.

For example, as shown in FIG. 5A (Example 10), a patient using a combinatorial therapeutic system with oliceridine and VRNT may experience a constant or linear decrease in pain over time. Some patients may experience a different response.

As shown in FIG. 5B (Example 10), a patient using a combinatorial therapeutic system with oliceridine and VRNT may experience a cyclical decrease and increase in pain levels over time with an overall decreasing pain level from a first time point to a second, later time point.

As an additional example (Example 12), a patient using a combinatorial therapeutic system with oliceridine and VRNT may experience a stepwise reduction in pain levels, as shown in FIG. 5C.

### Example 13

It should be appreciated that although FIGS. 5A-5C illustrate examples of a combinatorial therapeutic system/method with oliceridine and VRNT, the same or similar results should be expected for systemic, centrally and peripherally acting analgesic bupivacaine-meloxicam. Similarly, although VRNT is shown as the exemplary artificial sensory experience, the same or similar results may be expected for other artificial sensory experiences disclosed herein (e.g., 2D/3D/4D artificial sensory experiences, holography systems, other virtual reality systems, augmented reality systems, and/or mixed reality systems.)

It should be further appreciated that different patients may respond differently to the combinatorial therapy and may demonstrate a mixture of responses shown in FIGS. 5A-5C—or other responses. However, a significant proportion of patients utilizing the combinatorial systems/methods disclosed herein as intended will demonstrate an overall decrease in an amount or frequency of pain and/or longer lasting analgesic effect (or other benefits) and may, in some instances, cause a patient to no longer experience chronic pain and/or eliminate a patient's symptomatic pain.

### Examples 14 & 15

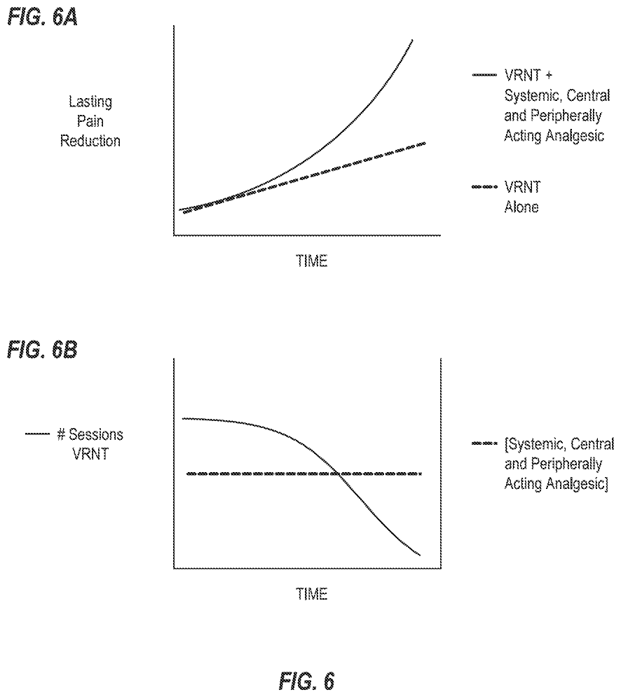

In some embodiments, the combinatorial therapeutic systems/methods may increase the effectiveness of the artificial sensory experience. For example, as shown in FIGS. 6A and 6B, a systemic, centrally and peripherally acting analgesic—when used in combination with an artificial sensory experience such as VRNT—can accelerate or increase lasting pain reduction (FIG. 6A) and/or reduce the number of trainings/sessions with the artificial sensory experience to achieve a beneficial reduction in pain (FIG. 6B). It should be appreciated that although FIGS. 6A and 6B illustrate the effects of combinatorial therapy using VRNT, other artificial sensory experiences may demonstrate the same or similar benefits. Without being bound to a particular theory, the results shown in FIGS. 6A and 6B may theoretically occur because the patient feels less pain while practicing VRNT and/or the systemic, centrally and peripherally acting analgesic can provide a beneficial amelioration of pain that is being visualized during VRNT. This may provide positive reinforcement to the psyche and/or willpower of the patient over their pain (e.g., the patient believes they are in control or can control the pain instead of being subject to it).

### Example Mechanisms of Synergy—VRNT Assisting Analgesics

Centrally and peripherally acting analgesics with systemic exposure can rely on psychological state (and placebo effect) for efficacy. VRNT increases the patients' own pain control, their feeling of self-efficacy, and their sense of optimism about pain outcomes, and reduces fear of pain, increasing the analgesic effectiveness of the analgesic and/or psychological perception thereof.

Centrally and peripherally acting analgesics with systemic exposure can provide pain relief or perception thereof. VRNT can be designed to create an audio-visual (plus tactile or other sensory) environment that mimics the effects of the analgesic. It could also exaggerate or accelerate these effects, which can lead the brain to believe in a much more powerful analgesic effect than the analgesic provides, leading to higher responder rates (more people responding to systemic, centrally and peripherally acting analgesics) and/or greater levels of analgesic effects.

Accordingly, methods and systems are provided for treating pain. The concepts and features described herein may be embodied in other specific forms without departing from their spirit or descriptive characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

1. A combinatorial therapeutic system, comprising:
a systemic, centrally and peripherally acting analgesic selected from oliceridine or bupivacaine-meloxicam;
an artificial sensory experience generator that includes a processor and computer-readable memory, the artificial sensory experience generator configured to implement an artificial sensory experience comprising one or more of 2D artificial sensory experience, 3D artificial sensory experience, 4D artificial sensory experience, holography, augmented reality, virtual reality, or mixed reality; and
an output device configured to produce sensory stimuli representative of a patient's symptom,
wherein the artificial sensory experience comprises a virtual representation of the patient's symptom, wherein the symptom includes pain.

2. The combinatorial therapeutic system as in claim 1, wherein the analgesic comprises oliceridine.

3. The combinatorial therapeutic system as in claim 1, wherein the analgesic comprises bupivacaine-meloxicam.

4. (canceled)

5. The combinatorial therapeutic system as in claim 1, wherein the artificial sensory experience comprises virtual reality distraction therapy.

6. The combinatorial therapeutic system as in claim 1, wherein the artificial sensory experience comprises virtual reality neuropsychological therapy (VRNT).

7. The combinatorial therapeutic system as in claim 6, wherein the VRNT comprises a 4D sensory experience that is implemented as a 3D visual stimulus and at least one additional sensory stimulus selected from auditory, tactile, olfactory, and gustatory.

8. The combinatorial therapeutic system as in claim 6, further comprising a 3D sensory experience selected from a 3D visual stimulus or a 2D visual stimulus and at least one additional sensory stimulus selected from auditory, tactile, olfactory, and gustatory.

9. The combinatorial therapeutic system as in claim 6, further comprising a 2D visual sensory stimulus.

10. A method for treating and/or mitigating pain in a patient, comprising:
the patient receiving a systemic, centrally and peripherally acting analgesic selected from oliceridine or bupivacaine-meloxicam;
the patient receiving an artificial sensory experience comprising one or more of 2D artificial sensory experience, 3D artificial sensory experience, 4D artificial sensory experience, holography, augmented reality, virtual reality, or mixed reality; and
the patient receiving sensory stimuli representative of a symptom,
wherein the artificial sensory experience comprises a virtual representation of the symptom, wherein the symptom includes pain.

11-13. (canceled)

14. The method as in claim 10, wherein the artificial sensory experience comprises virtual reality distraction therapy.

15. The method as in claim 10, wherein receiving an artificial sensory experience comprises receiving virtual reality neuropsychological therapy (VRNT), wherein receiving the analgesic and receiving VRNT synergistically interact to reduce one or more of pain or analgesic usage.

16. The method as in claim 15, wherein the VRNT comprises a 4D sensory experience that is implemented as a 3D visual stimulus and at least one additional sensory stimulus selected from auditory, tactile, olfactory, and gustatory.

17. (canceled)

18. The method as in claim 15, wherein receiving an artificial sensory experience further comprises receiving a 3D sensory experience selected from: (i) a 3D visual stimulus or (ii) a 2D visual stimulus and at least one additional sensory stimulus selected from auditory, tactile, olfactory, and gustatory.

19. The method as in claim 18, wherein the 3D sensory experience is received after receiving the VRNT and wherein the 3D sensory experience maintains the reduction of one or more of pain or analgesic usage.

20. (canceled)

21. The method as in claim 15, wherein receiving an artificial sensory experience further comprises receiving a 2D visual sensory stimulus and wherein the 2D sensory experience maintains the reduction of one or more of pain or analgesic usage.

22. (canceled)

23. The method as in claim 21, wherein the 2D sensory experience is received after receiving the VRNT.

24. (canceled)

25. The system as in claim 1, wherein the artificial sensory experience generator (104) is configured to implement the virtual representation of the patient's symptom (120) based on user input received via an input device (110), the user input being incorporated into a symptom visualization protocol (114) which causes output device (112) to produce sensory stimuli representative of the symptom (120).

26. The system as in claim 8, wherein the artificial sensory experience generator (104) is configured to implement the virtual representation of the patient's symptom (120) by receiving a user drawing of the virtual representation of the symptom (120) that is then displayed within the artificial sensory environment.

27. The system as in claim 9, wherein receiving the user drawing comprises receiving user selection of a symptom template.

28. The system as in claim 1, wherein the artificial sensory experience generator (104) generates the virtual representation of the symptom (120) by providing a shape located on or within an avatar representing the user.

29. The system as in claim 11, wherein the shape is located on or within the avatar at a location associated with the real-world symptom of the user.

30. The system as in claim 12, wherein the artificial sensory experience generator (104) additionally generates other sensory stimuli representative of the symptom (120), the other sensory stimuli comprising audio stimuli.

31. The system as in any one of claims 11-13, wherein the artificial sensory experience further comprises an alleviation protocol (116) that implements a change to the virtual representation of the symptom (120) representing an improvement in the symptom (120).

32. The system as in claim 14, wherein the alleviation protocol (116) comprises modifying both visual and audio sensory stimuli associated with the symptom (120). [See paragraph 0036 of the PCT Application]
